(19) United States
(12) Patent Application Publication
(10) Pub. No.: US 2009/0081249 A1
(43) Pub. Date: Mar. 26, 2009

(54) Bi-Functional Polymer-Attached Inhibitors of Influenza Virus

(75) Inventors: Jayanta Haldar, South Boston, MA (US); Luis Alvarez de Cienfuegos, Boston, MA (US); Alexander M. Klibanov, Boston, MA (US); Jianzhu Chen, Lexington, MA (US)

Correspondence Address: Pabst Patent Group LLP, 1545 PEACHTREE STREET NE, SUITE 320, ATLANTA, GA, 30309, US

(73) Assignee: Massachusetts Institute of Technology

(21) Appl. No.: 12/197,452

(22) Filed: Aug. 25, 2008

Related U.S. Application Data
(60) Provisional application No. 60/968,213, filed on Aug. 27, 2007.

(51) Int. Cl. A61K 39/385; A61K 47/48; A61P 31/16

(52) U.S. Cl. 424/193.1

(57) ABSTRACT

Antimicrobial compositions containing two or more antiviral agents coupled to a polymer and methods of making and using the compositions, are described herein. In one embodiment, two or more antiviral agents are covalently coupled to the polymer. Suitable antiviral agents include, but are not limited to, sialic acid, zanamivir, oseltamivir, amantadine, rimantadine, and combinations thereof. The polymer is preferably a water-soluble, biocompatible polymer. Suitable polymers include, but are not limited to, poly(isobutylene-alt-maleic anhydride) (PIBMA), poly(aspartic acid), poly(l-glutamic acid), polylysine, poly(acrylic acid), plyaginic acid, chitosan, carboxymethyl cellulose, carboxymethyl dextran, polyethyleneimine, and blends and copolymers thereof. In another embodiment, the compositions contain a physical mixture of polymer containing one antiviral agent and polymer containing a second antiviral agent. The compositions can be formulated for enteral or parenteral administration. Suitable oral/intranasal dosage forms include, but are not limited to, tablets, capsules, solutions, suspensions, emulsions, syrups, and lozenges. Suitable dosage forms for parenteral administration include, but are not limited to, solutions, suspensions, and emulsions. The compositions described herein are effective at treating a variety of infections, including viral infections such as influenza, while inhibiting or preventing the development of microbial resistance.

### CROSS-REFERENCE TO RELATED APPLICATIONS

This application claims benefit of and priority to U.S. Ser. No. 60/968,213, filed on Aug. 27, 2007, which is incorporated by reference in its entirety.

### GOVERNMENT SUPPORT

The United States government may have certain rights in this technology by virtue of financial support by the U.S. Army through the Institute for Soldier Nanotechnologies at MIT under Contract DAAD-19-02-D-0002 with the Army Research Office and NIH grants to Jianzhu Chen A156267 (6895481) and AI074443 (6915739).

### FIELD OF THE INVENTION

This invention is generally in the field of polymer compositions which exhibit virucidal and/or virustatic activity.

### BACKGROUND OF THE INVENTION

Influenza A virus causes epidemics and pandemics in human populations, inflicting enormous suffering and economic loss. Currently, two distinct strategies, vaccines and small molecule therapeutics, are used to try to control the spread of the virus. Vaccination offers limited protection, however, and is hampered by several logistical challenges, such as accurately predicting future circulating strains, production of sufficient quantities of vaccines for large populations in a short period of time, and administering the vaccine to populations which are at risk.

With respect to small molecule therapeutics, there are currently four antiviral drugs for the treatment and/or prevention of influenza: amantadine, rimantadine, zanamivir, and oseltamivir. Although these drugs may reduce the severity and duration of influenza infections, they have to be administered within 24-48 hours after the development of symptoms in order to be effective. Further, the emergence of stable and transmissible drug-resistant influenza strains can render these drugs ineffective.

To overcome drug resistance, combination therapies, which contain two or more drugs that simultaneously interfere with different vital processes of a microbe, have to be used. Amantadine and rimantadine inhibit the M2 ion channel protein, whereas zanamivir and oseltamivir inhibit the neuraminidase enzyme (NA). Unfortunately, because most of the circulating influenza viruses are already resistant to the M2 inhibitors, traditional combination therapies involving these four drugs have little added value for influenza control. There exists a need for antiviral compositions that are effective in treating viral infections while inhibiting or preventing the development of microbial resistance

It is an object of the invention to provide antiviral compositions that are effective in treating viral infection, such as influenza, while inhibiting or preventing the development of viral resistance, and methods of making and using thereof.

### SUMMARY OF THE INVENTION

Antiviral compositions containing one or more antiviral agents coupled to a polymer and methods of making and using the compositions, are described herein. The one or more antiviral agents are covalently coupled to the polymer, and thereby prevent or decrease development of drug resistance. Suitable antiviral agents include, but are not limited to, sialic acid, zanamivir, oseltamivir, amantadine, rimantadine, and combinations thereof. The polymer is preferably a water-soluble, biocompatible polymer. Suitable polymers include, but are not limited to, poly(isobutylene-alt-maleic anhydride) (PIBMA), poly(aspartic acid), poly(glutamic acid), polylysine, poly(acrylic acid), plyaginic acid, chitosan, carboxymethyl cellulose, carboxymethyl dextran, polyethyleneimine, and blends and copolymers thereof. In another embodiment, the compositions contain a physical mixture of polymer containing one antiviral agent and polymer containing a second antiviral agent. In one embodiment, the composition contains two antimicrobial agents, such as sialic acid and zanamivir, coupled to PIBMA. In another embodiment, the compositions contains a physical mixture of a first antimicrobial agent, such as sialic acid, coupled to PIBMA and a second antimicrobial agent, such as zanamivir, coupled to PIBMA.

The concentration of the antiviral agent(s) is from about 5% to about 25% by weight of the polymer. In one embodiment the concentration of each antiviral agent is independently 5% by weight of the polymer, 8% by weight of the polymer, 10% by weight of the polymer, 15% by weight of the polymer, 18% by weight of the polymer, 20% by weight of the polymer, or 25% by weight of the polymer.

The compositions can be formulated for enteral or parenteral administration.

Suitable oral dosage forms include, but are not limited to, tablets, capsules, solutions, suspensions, emulsions, syrups, and lozenges. Suitable dosage forms for intranasal include, but are not limited to, solutions, suspensions, powders and emulsions. Suitable dosage forms for parenteral administration include, but are not limited to, solutions, suspensions, and emulsions.

The compositions described herein are effective at treating a variety of viral infections, such as influenza, respiratory syncythial virus, rhinovirus, human metaneurovirus, and other respiratory diseases, while inhibiting or preventing the development of resistance. For example, a conjugate containing poly(isobuylene-alt-maleic anhydride), 10% zanamivir, and 10% sialic acid had an IC₅₀ value of 7 nM, which is a 90-fold increase compared to monomeric zanamivir. In another example, an equimolar combination of the monofunctional agents (PIBMA-SA+PIBMA-ZA) was at least an order of magnitude more potent inhibitor of influenza A viruses, whether of the wild-type or mutant strains, than monofunctional multivalent agents.

### BRIEF DESCRIPTION OF THE DRAWINGS

FIG. 1 shows the reaction scheme for converting sialic acid to the activated derivative of zanamivir.

FIG. 2 shows the reaction scheme for coupling the activated derivative of zanamivir to poly(isobutylene-alt-maleic anhydride).

FIG. 3 shows the reaction scheme for the synthesis of the O-glycoside of sialic acid.

FIG. 4 shows the reaction scheme for the coupling of the O-glycoside of sialic acid to poly(isobutylene-alt-maleic anhydride).

FIG. 5 shows the reaction scheme for the coupling of both the activated derivative of zanamivir and the O-glycoside of sialic acid to poly(isobutylene-alt-maleic anhydride).

FIG. 6 shows the inhibition of influenza virus (Victoria) infection by PIMBA-ZA-SA in MDCK cell culture. Wells 1-5 show the result of treatment with decreasing concentration of PIBMA-ZA-SA from 500 ng/ml to 0.05 ng/ml. Well 6 contains PBS (control).

FIG. 7 is a graph showing the inhibition of influenza virus production in mouse lungs by PIMBA-ZA-SA.

### I. DEFINITIONS

“Virucidal”, as used herein, means capable of neutralizing or destroying a virus.

“Virustatic, as used herein, means inhibiting the replication of viruses.

“Biocompatible”, as used herein, means the material does not cause injury, or a toxic or immunologic reaction to living tissue.

“Water soluble polymer”, as used herein, means a polymer having at least some appreciable solubility in water or monophasic aqueous-organic mixtures, e.g., over 1 mg/liter at room temperature.

“IC₅₀”, as used herein, means the concentration of polymer-bound drug to reduce the number of plaques by 50% compared to the number of plaques observed in the absence of polymer-bound rug, both determined by a plaque reduction assay under the same conditions. The IC₅₀ measures the prevention of infection.

“Inhibit or decrease drug resistance”, as used herein, refers to lowering incidence of the emergence of resistant virus or inhibiting influenza viruses that are already resistant to antiviral drugs, such as zanamivir.

### II. COMPOSITIONS

Antiviral compositions containing one or more antiviral agents covalently coupled to a water-soluble polymer are described herein. In one embodiment, two or more different antiviral agents are coupled to a water soluble polymer. In another embodiment, the composition contains a blend of a first water-soluble polymer coupled to a first antiviral agent and a second water-soluble polymer coupled to a second antiviral agent.

A. Antiviral Agents

Any antiviral agent can be used provided that the agent retains some of its activity upon coupling to the polymer. Exemplary classes of antiviral drugs include, but are not limited to, neuraminidase inhibitors, M2 inhibitors, proteinase inhibitors, inosine 5′-monophosphate (IMP) dehydrogenase (a cellular enzyme) inhibitors, viral RNA polymerase inhibitors, and siRNAs. Suitable agents include, but are not limited to, sialic acid, zanamivir, oseltamivir, amantadine, rimantadine, and combinations thereof. Zanamivir and oseltamivir inhibit the neuraminidase enzyme (NA), while amantadine and rimantadine inhibit the M2 ion channel protein.

Zanamivir is a relatively small molecule (MW 1,000 Da) that binds to the catalytic site of viral NA to inhibit its activity. Polymers coupled to zanamivir through a covalent linker can be prepared in such a way that the zanamivir moiety in the polymer is still able to bind to the catalytic site and inhibit NA activity. Such polymer-bound antiviral agents should be effective in both inhibiting viral infections, such as influenza, and preventing the emergence of drug resistant viruses.

Without being bound by any one theory, it is hypothesized that polymer-bound antiviral agents will be more potent inhibitors than monomer antiviral agent due to multivalent binding. The influenza virion contains 30-50 NA and 300-500 HA molecules. Thus, the presence of multiple zanamivir and sialic (SA) moieties attached to the same polymer backbone can simultaneously bind to multiple NA and hemagglutinin (HA) on the same virion. This significant increase in the avidity between polymer-bound antiviral moiety and NA/HA should make the polymer-antiviral agent complex a more potent competitive inhibitor. Secondly, because of multivalent binding, the polymer-bound antiviral agent should remain a potent inhibitor of NA/HA even if changes in NA/HA significantly weaken the binding of monomeric antiviral agent to the enzyme's active site. For example, zanamivir binds to the active site of NA with an affinity constant of 10⁻¹⁰ to 10⁻⁹ M (0.1-1.0 nM). Even if the binding affinity is reduced by 10⁶- to 10⁴-fold, the conjugate should still be a potent inhibitor provided that more than three zanamivir moieties attached to the same polymer backbone bind to NA on the same virion at the same time. This is supported by the fact that zanamivir still binds to the catalytic site of NA of most zanamivir resistant viruses (IC₅₀ of 15 to 645 nM). Finally, the binding of a large polymer to multiple NA molecules could create steric hindrance or viral aggregates that interfere with viral infection in addition to the viral release from infected cells.

Coupling two or more other inhibitors, which inhibit influenza virus through a different target, to the same polymer backbone and/or combination of monofunctional polymer-attached ligands should more effectively suppress viral resistance. For example, during influenza virus infection, bonding of hemagglutinin (HA) to sialic acid (SA) residues of glycoproteins on the surface of the cell is critical for viral entry into the cell. Since SA is the cellular receptor for influenza virus, the use of SA itself may help to suppress viral resistance because a viral HA that does not bind sialic acid may have reduced ability to infect host cells.

Both zanamivir and sialic acid exert their effects by binding to particular targets (NA and HA, respectively) on the virion. Therefore, binding these agents to the same polymer backbone should result in a composition that does not need to be taken into the cell to exert its inhibitory effect. Bi-functional polymers containing either both zanamivir and sialic acid covalently bound to the same polymer backbone or a physical mixture of polymer containing zanamivir and polymer containing sialic acid, may prove to be particularly effective in preventing the emergence of drug-resistant viruses. Zanamivir and sialic acid inhibit influenza virus through different targets and therefore should benefit from combination therapy. Moreover, due to multivalent binding, polymeric inhibitors may remain effective against virus which are resistant to monomeric inhibitors.

The concentration of the antiviral agent is from about 5% to about 25% by weight of the polymer. In one embodiment, the concentration of each antiviral agent is independently 5% by weight of the polymer, 8% by weight of the polymer, 10% by weight of the polymer, 15% by weight of the polymer, 18% by weight of the polymer, 20% by weight of the polymer, or 25% by weight of the polymer.

B. Polymers

The two or more antimicrobial agents can be coupled to any water-soluble, biocompatible polymer. In one embodiment, the two or more antimicrobial agents are coupled to the same polymer. In another embodiment, the composition contains a physical mixture of a first antimicrobial agent coupled to a first water-soluble, biocompatible polymer and a second antimicrobial agent coupled to a second water-soluble, biocompatible polymer. The polymers may be the same polymer (i.e., have the same chemical composition and molecular weight) or different polymers (i.e., different chemical compositions and/or molecular weights).

The polymer is preferably non-toxic and non-immunogenic and is readily excreted from living organisms. In one embodiment, the polymer is biodegradable. Preferably, the antiviral agent(s) are coupled to the polymer via a functional group which is shown not to participate in the binding of the agent to the virus. For example, X-ray crystal structures of zanamivir bound to influenza NA show that the 7-hydroxyl group of the sugar has no direct contact with NA and therefore the attachment of the agent to the polymer via the 7-position should not disrupt the binding interaction. The 7-hydroxyl group can also be converted to other reactive functional groups, such as amino groups or sulfhydryl groups. Therefore, polymers containing functional groups which react with hydroxy, amino, or sulfhydryl groups or groups which are capable of being converted to functional groups which react with hydroxy, amino, or sulfhydryl groups can be used to prepare the compositions described herein. Alternatively, the polymer can contain nucleophilic groups, such as hydroxy, amino, or thiol groups, which react with electrophilic groups on the antimicrobial agent.

Suitable polymers include, but are not limited to, poly(isobutylene-alt-maleic anhydride) (PIBMA), poly(aspartic acid), poly(glutamic acid), polylysine, poly(acrylic acid), plyaginic acid, chitosan, carboxymethyl cellulose, carboxymethyl dextran, polyethyleneimine, and blends and copolymers thereof. The polymers typically have a molecular weight of 1,000 to 1,000,000 Daltons, preferably 10,000 to 1,000,000 Daltons. In one embodiment, the composition contains two antimicrobial agents, such as sialic acid and zanamivir, coupled to PIBMA. In another embodiment, the compositions contains a physical mixture of a first antimicrobial agent, such as sialic acid, coupled to PIBMA and a second antimicrobial agent, such as zanamivir, coupled to PIBMA.

### III. METHOD OF MANUFACTURE

The compositions described herein can be prepared by covalently attaching antiviral agents, or derivative thereof, to a water-soluble, biocompatible polymer. For example, the antiviral agents to be coupled to the polymer are activated using a variety of chemistries known in the art to form reactive derivatives. The reactive derivative of the antimicrobial agent is reacted with the polymer to covalently link the antiviral agents to the polymer. The reactive derivative can contain a nucleophilic or electrophilic group which reacts with an electrophilic group or nucleophilic group on the polymer.

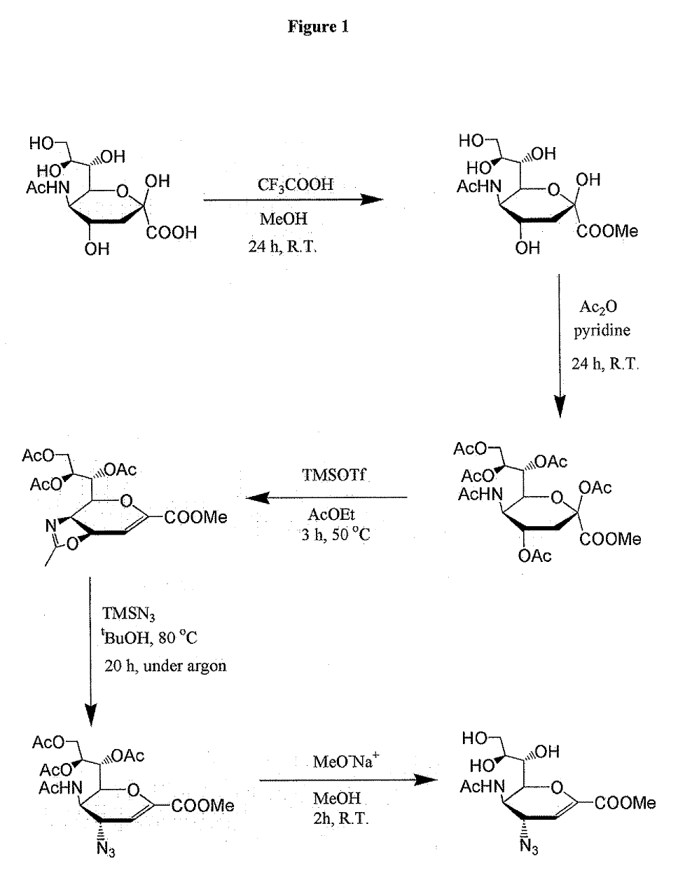

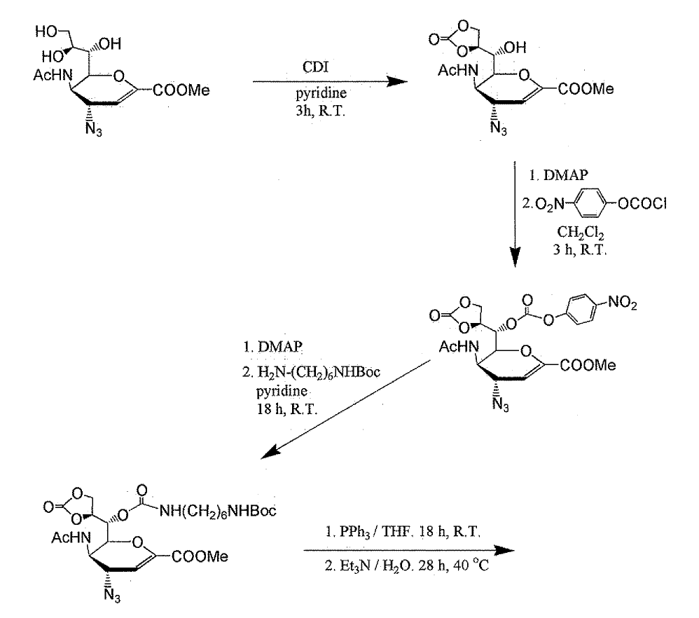

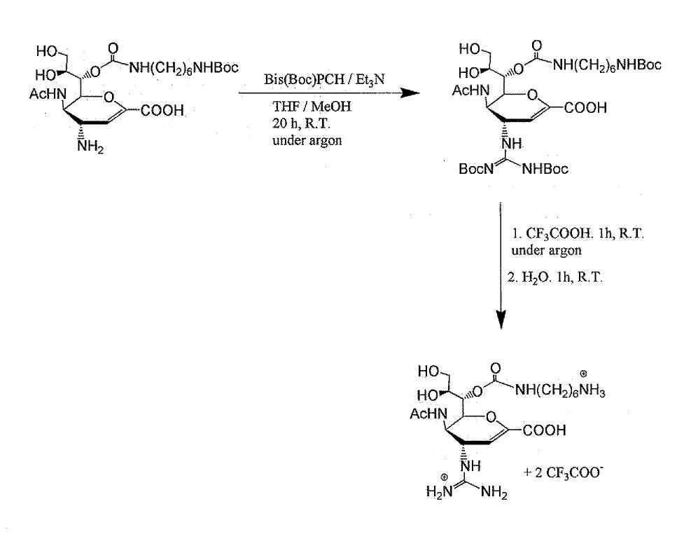

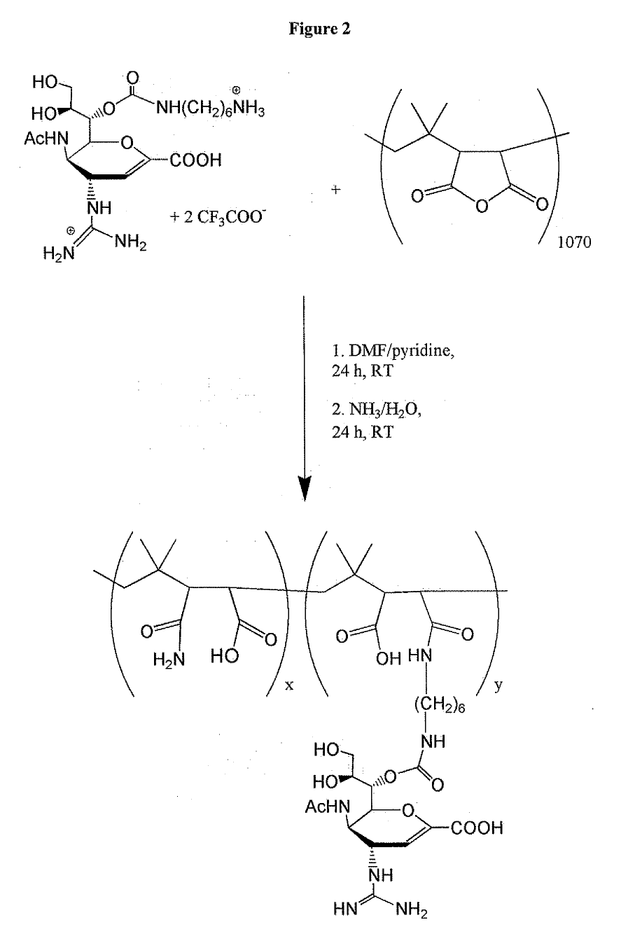

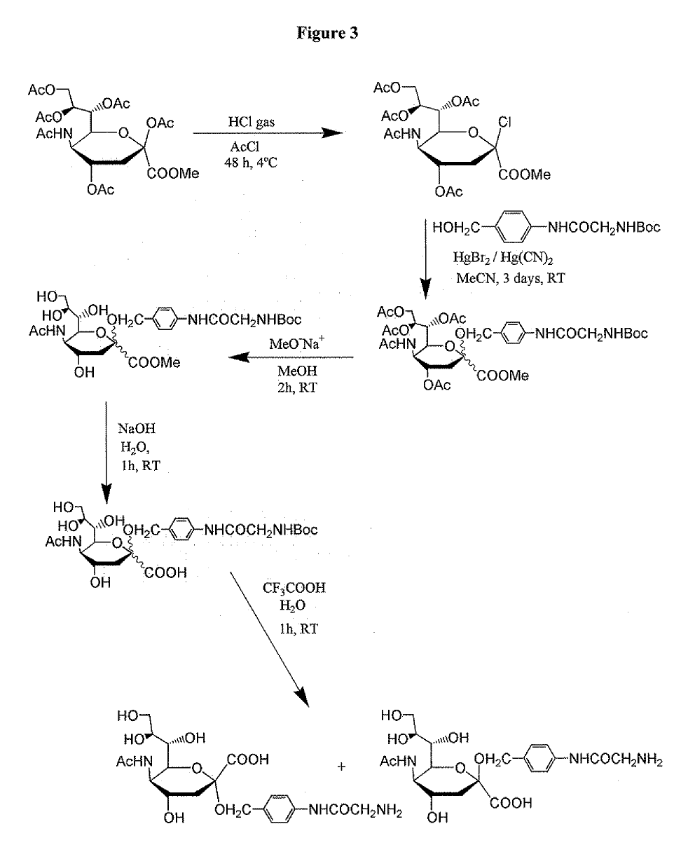

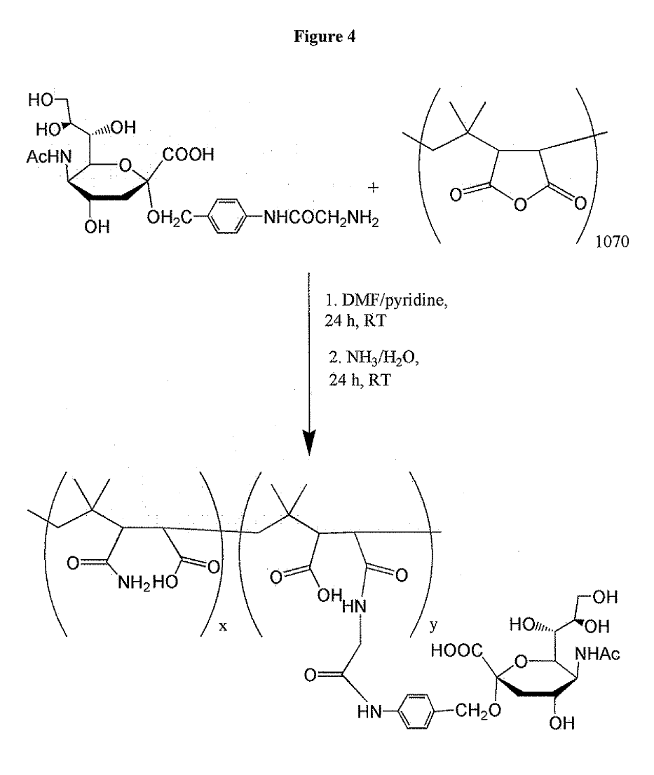

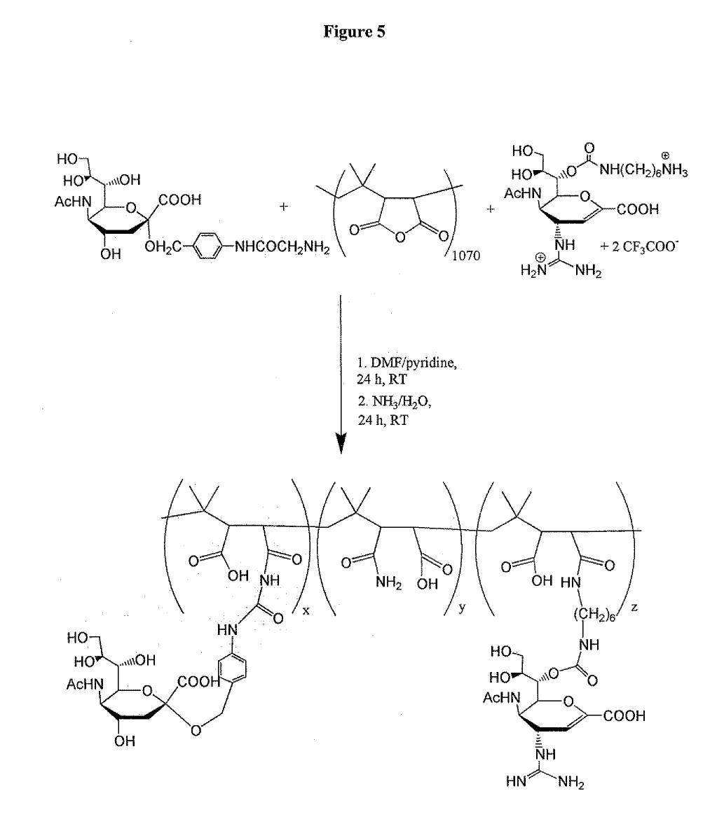

In one embodiment, sialic acid is converted to an activated derivative of the antiviral agent zanamivir. FIG. 1 shows the reaction scheme for converting sialic acid to the activated derivative of zanamivir. FIG. 2 shows the reaction scheme for coupling the activated zanamivir to PIBMA through the 7-hydroxyl group of the sugar in zanamivir. X-ray crystal structures of zanamivir bound to influenza NA show that the 7-hydroxyl group of the sugar has no direct contact with NA and therefore the attachment of the agent to the polymer via the 7-position should not disrupt the binding interaction. FIG. 3 shows the reaction scheme for the synthesis of the O-glycoside of sialic acid. The O-glycoside of sialic acid is coupled to PIBMA as shown in FIG. 4, FIG. 5 shows the reaction scheme for the simultaneous coupling of both activated zanamivir derivative and the O-glycoside of sialic acid to PIBMA.

The dosage to be administered can be readily determined by one of ordinary skill in the art and is dependent on the age and weight of the patient and the infection to be treated. The amount of antiviral agent molecules to be coupled to the polymer is dependent upon the number of reactive groups on the polymer. For example, PIBMA having a weight average molecular weight of 165 kDa, has approximately 1,070 repeating units. The average number of sialic acid residues per polymer chain is 5%, 10%, 12%, 16%, and 33% occupancy is 53, 106, 128, 171, and 353, respectively. Similarly, PIBMA (165 kDa) containing 5-25% zanamivir contains 53-267 zanamivir moieties per polymer chain. The PIBMA polymeric chain bearing 10% sialic acid and 10% zanamivir contains some 106 units each of the two sugar moieties.

### IV. METHODS OF USE AND ADMINISTRATION

The compositions described herein can be used to treat and/or prevent infections in a mammal, such as a human. Infections to be treated include, but are not limited to, viral infections, such as influenza; bacterial infections; fungal infections; parasitic infections; or combinations thereof. The compositions described herein can be formulated for parenteral or enteral administration. In one embodiment, the infection is a viral infection, such as avian or human influenza A or B. The compositions are effective against wild-type or mutant avian and human influenza viruses.

A. Dosage Forms

The compositions described herein can be formulated for enteral, parenteral, or topical formulation. In one embodiment, the compositions are formulated for enteral or parenteral administration. The formulations may contain one or more pharmaceutically acceptable excipients, carriers, and/or additives. Methods for preparing enteral and parenteral dosage forms are described in Pharmaceutical Dosage Forms and Drug Delivery Systems, 6^{th} Ed., Ansel et al., Williams and Wilkins (1995).

a. Enteral Dosage Forms

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, pH-modifying agents, preservatives, binders, lubricants, disintegrators, fillers, and coating compositions.

Carrier also includes all components of the coating composition which may include plasticizers, pigments, colorants, stabilizing agents, and glidants. Delayed release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta,-alanine, sodium N-lauryl-.beta.-iminodipropionate, myristoamphoacetate, lauryl betaine and lauryl sulfobetaine.

b. Parenteral Dosage Forms

Suitable parenteral dosage forms include, but are not limited to, solutions, suspension, and emulsions. Formulations for parenteral administration may contain one or more pharmaceutically acceptable excipients including, but not limited to, surfactants, salts, buffers, pH modifying agents, emulsifiers, preservatives, anti-oxidants, osmolalityltonicity modifying agents, and water-soluble polymers.

The emulsion is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal.

Other dosage forms include intranasal dosage forms including, but not limited to, solutions, suspensions, powders, and emulsions. The dosage forms may contain one or more pharmaceutically acceptable excipients and/or carriers. Suitable excipients and carriers are described above.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Synthesis of Poly(isobutylene-alt-maleic anhydride)-Zanamivir (PIMBA-ZA) Conjugates

Synthesis of Zanamivir Derivatives

The monomeric zanamivir analogue was synthesized using the following published procedures with some modifications: a) Chandler, M., M. J. Bamford, R. Conroy, B. Lamont, B. Patel, V. K. Patel, I. P. Steeples, R. Storer, N. G. Weir, M. Wright, and C. Williamson. 1995. Synthesis of the potent influenza neuraminidase inhibitor 4-guanidino Neu5Ac2en. X-Ray molecular structure of 5-acetamido-4-amino-2,6-anhydro-3,4,5-trideoxy-D-erytro-L-gluco-nononic acid. J. Chem. Soc. Perkin Trans. 1:1173-1180. b) Andrews, D. M., P. C. Chemy, D. C. Humber, P. S. Jones) S. P. Keeling, P. F. Martin, C. D. Shaw, and S. Swanson. 1999. Synthesis and influenza virus sialidase inhibitory activity of analogues of 4-Guanidino-Neu5Ac2en (Zanamivir) modified in the glycerol side-chain. Eur. J. Med. Chem. 34:563-574. The synthesis is shown in FIG. 1.

Synthesis of PIMBA-Bearing Zanamivir Derivatives

28 mg (0.04 mmol) of the monomeric zanamivir derivative prepared above was added to a solution of PIMBA (100 mg, 0.65 mmol on the basis of the monomer) in dry dimethylformamide (DMF, 10 mL) and pyridine (0.5 mL). The reaction mixture was stirred at room temperature for 24 hours and then quenched with NH₄OH (28%, 10 mL) solution at room temperature for 24 hours. The resulting mixture was dialyzed (molecular weight cutoff of 3,500 Daltons) against distilled water for 48 hours and then lyophilized to yield a white powder. The polymer contained 5% ZA. Polymers containing 8% ZA, 18% ZA, and 25% ZA were also prepared. The reaction scheme for the formation of PIMBA-ZA is shown in FIG. 2. The amount of zanamivir derivative coupled to the polymer backbone was quantified by ¹H-NMR and the yield was above 80%.

¹H-NMR (400 MHz, D₂O+MeOD): δ 5.60-5.50 (1H, m, H-3); 4.50-4.30 (2H, m, H-4,6); 4.15-3.90 (2H, m, H-5,8); 3.65-3.35 (2H, m, H-9ₐ,9_{b}); 3.10-2.55 (5H, m, 4H-linker, 1H polymer); 2.55-2.20 (1H, m, 1H polymer); 2.20-1.75 (4H, m, CH₃CON, 1H polymer); 1.75-1.25 (9H, 8H-linker, 1H polymer); and 1.25-0.75 (6H, m, 6H polymer).

### Example 2

### Antiviral activity of PIMBA-ZA conjugates

To determine the antiviral activity of PIMBA-ZA, plaque reduction assays were performed. The assay was conducted by mixing 125 μl of ten-fold series dilutions of the inhibitors with an equal volume of influenza A/Victoria/3/75 (H3N2) in phosphate-buffered solution (“PBS”) (800 plaque forming unit (pfu)/mL). Following incubation at room temperature for one hour, 200 μl of the reaction mixture was added to confluent Madin-Darby canine kidney (“MDCK”) cells in 6-well cell culture plates and incubated at room temperature for one hour. After incubation, the solution was removed by aspiration. The cells were then overlaid with 2 ml of the F12 plaque medium and incubated at 37° C. for 3 days. The cultures were fixed with 1% formaldehyde for one hour at room temperature, the cells were stained with a 1% crystal violet dye solution, and the plaques were counted. As controls, no inhibitor, monomeric zanamivir derivative, or bare PIMBA were used. By comparing the number of plaques with that observed in the control experiment (no inhibitor), the concentrations of the inhibitors required to reduce the number of viral plaques by 50% (IC₅₀) were calculated. For easy comparison, the IC₅₀ values were calculated as concentrations of either polymer or zanamivir derivative. The results are shown in Table 1.

TABLE 1
IC₅₀ values of various PIBMA-ZA derivatives

| | IC₅₀ | | |
|---|---|---|---|
| | PIMBA | Sugar | |
| Compounds | (ng/ml) | (ng/ml) | (nM) |
| PIBMA | (2.1 ± 0.5) × 10⁴ | — | — |
| Zanamivir | — | (4.4 ± 0.5) × 10² | (6.3 ± 0.8) × 10² |
| derivative | | | |
| PIBMA-ZA | 29 ± 20 | 3.6 ± 3 | 7.6 ± 5 |
| (5%) | | | |
| PIBMA-ZA | 21 ± 20 | 3.8 ± 3.6 | 7.9 ± 7 |
| (8%) | | | |
| PIBMA-ZA | 4.7 ± 1 | 1.6 ± 0.6 | 3.3 ± 0.1 |
| (18%) | | | |
| PIBMA-ZA | 4.9 ± 1 | 2.1 ± 0.4 | 4.3 ± 0.8 |
| (25%) | | | |

As shown in Table 1, PIBMA itself has little detectable antiviral activity. The IC₅₀ of zanamivir derivative is approximately 630 nM. In contrast, the IC₅₀ values of PIBMA-ZA are around 5 nM (depending on the percentage of zanamivir conjugation), representing some 100 fold improvement in efficacy. Moreover, variation in the percentage of zanamivir conjugated to the polymer has only a modest effect of the IC₅₀ values. These results suggest that water-soluble PIBMA can be readily conjugated to a zanamivir derivative, wherein the zanamivir remains effective. Note that 5-25% zanamivir content corresponds to an average of 53 to 267 zanamivir moieties per polymer chain. As there are only 35-50 NA molecules per virion, this is a significant excess of zanamivir moieties. It is possible that a lower zanamivir content may promote aggregate formation and therefore yield a more potent PIBMA-ZA inhibitor.

### Example 3

### Synthesis of Poly(isobutylene-alt-maleic anhydride)-sialic acid (PIBMA-SA) conjugates

Synthesis of an O-Glycoside of Sialic Acid

Sialic acid was coupled to a linker using the following published procedures: a) Baumberger, F., A. Vasella, and R. Schauer. 1986. 4-methylumbelliferyl 5-acetamido-3,4,5-trideoxy—D-manno-2-nonulopyranosidonic Acid: Synthesis and Resistance to Bacterial Sialidases. Helvetica Chimica Acta 69:1927-1935, b) Warner, T. G., and L. Laura. 1988. An azidoaryl thioglycoside of sialic acid. A potential photoaffinity probe of sialidases and sialic acid-binding proteins. Carbohydrate Research 176:211-218. c) Byramova, N. E., L. V. Mochalova, I. M. Belyanchikov, M. N. Matrosovich, and N. V. Bovin. 1991. Synthesis of sialic acid pseudopolysaccharides by coupling of spacer-connected Neu5Ac with activated polymer. J. Carbohydr. Chem. 10:691-700. The synthesis is shown in FIG. 3.

Synthesis of Polymers of O-Glycoside of Sialic Acid

Conjugation of the O-glycoside of sialic acid prepared above follows the same methodology described above for the conjugation of zanamivir derivative to PIBMA. The polymer contained 5% SA. Polymers containing 10% SA, 12% SA, and 16% SA and 33% SA were also prepared. The reaction scheme for the formation of PIBMA-SA is shown in FIG. 4. The amount of sialic acid derivative coupled to the polymer backbone was quantified by ¹H-NMR and the yield was above 80%.

¹H-NMR (400 MHz, D₂O+MeOD): δ 7.50-7.20 (4H, m, aromatics); 4.40 (1H, m, CH₂Ph); 4.00-3.50 (9H, m, CH₂N, H-4, 5, 6, 7, 8, 9ₐ, 9_{b}); 2.90-2.60 (2H, m, H-3_{eq}, 1H polymer); 2.60-2.20 (1H, m, 1H polymer); 2.20-1.75 (4H, m, CH₃CON, 1H polymer), 1.75-1.25 (2H, H-3ₐₓ, 1 polymer); and 1.25-0.75 (6H, m, 6H polymer).

### Example 4

### Antiviral Activity of PIMBA-SA Conjugates

To determine the antiviral activity of PIBMA-SA conjugates, plaque reduction assays were performed as described above for PIBMA-ZA conjugates. The results are shown in Table 2.

TABLE 2
IC₅₀ values of various PIBMA-SA derivatives

IC₅₀

| | PIMBA | Sugar | |
|---|---|---|---|
| Compounds | (ng/ml) | (ng/ml) | (nM) |
| PIBMA | (2.1 ± 0.5) × 10⁴ | — | — |
| Sialic acid | — | >>5 × 10⁵ | >>10⁶ |
| derivative | | | |
| PIBMA-SA (5%) | (4.4 ± 0.6) × 10² | 54 ± 7 | (1.1 ± 0.2) × 10² |
| PIBMA-SA | (2.2 ± 0.3) × 10² | 48 ± 14 | (1.0 ± 0.1) × 10² |
| (10%) | | | |
| PIBMA-SA | 41 ± 5 | 10 ± 2 | 22 ± 3 |
| (12%) | | | |
| PIBMA-SA | 4 ± 1 | 1.3 ± 1 | 3 ± 1 |
| (16%) | | | |
| PIBMA-SA (33%) | 17 ± 1 | 8 ± 1 | 17 ± 1 |

As shown in Table 2, the sialic acid modified with the linker had no detectable antiviral activity. The IC₅₀ values of PIBMA-SA range from 114 nM to 3 nM, depending on the percentage of SA conjugated to the polymer. With 5% or 10% of the available sites in the polymer conjugated to the sialic acid derivative, the IC₅₀ value is around 100 nM. With 12% occupancy, the IC₅₀ value dropped some 5 fold to 22 nM. With 16% occupancy, there was an additional 7-fold decrease in the IC₅₀ value to 3 nM. However, with 33% occupancy, the IC₅₀ value rose to 17 nM. These variations in the IC₅₀ value as a function of sialic acid content suggests that there is an optimum amount of SA conjugation.

The weight average molecular weight of the PIBMA backbone is 165 kDa which correlates to 1,070 repeating units. The average numbers of sialic acid residues per polymer chain at 5%, 10%, 12%, 16%, and 33% occupancy are 53, 106, 128, 171, and 353, respectively. As each influenza virion has 350-500 HA molecules, there does not appear to be a simple correlation between the IC₅₀ values and the number of sialic acids conjugated to the polymer chain.

### Example 5

### Synthesis of PIBMA Bearing Both Zanamivir and Sialic Acid

Conjugation of both sialic acid and zanamivir derivatives to the same PIBMA polymer can be done using the same methodologies described above for conjugating zanamivir to PIBMA. O-glycoside of sialic acid was added to a solution of PIBMA in dry DMF and pyridine. The zanamivir analogue was added and the reaction mixture was quenched with NH₄OH. The resulting solution was dialyzed against distilled H₂O and lyophilized to yield a white powder. The reaction scheme for the formation of PIBMA-ZA-SA is shown in FIG. 5. The amount of sialic acid and zanamivir coupled to the polymer backbone was quantified by ¹H-NMR and the yield was above 80%.

¹H-NMR (400 MHz, D₂O+MeOD): δ 7.50-7.20 (4H, m, aromatics); δ 5.60-5.50 (1H, m, 1H-3); 4.50-4.30 (3H, m, CH₂P(SA), H-2(ZA)); 4.20-3.90 (2H, m, H-5,8(ZA)); 3.90-3.40 (11H, m, CH₂N, H-4, 5, 6, 7, 8, 9ₐ, 9_{b}(SA), H-9ₐ, 9_{b}(ZA)); 3.10-2.50 (6H, m, H-3_{eq}(SA), 4H-linker(ZA), 1H polymer); 2.60-2.20 (1H, m, 1H polymer); 2.20-1.80 (7H, m, CH₃CON(SA), CH₃CON(ZA), 1M polymer), 1.75-1.25 (10, H-3ₐₓ(SA), 8H-linker(ZA), 1H polymer); and 1.25-0.75 (6H, m, 6H polymer).

### Example 6

### Antiviral Activity of PIMBA-ZA-SA Conjugates

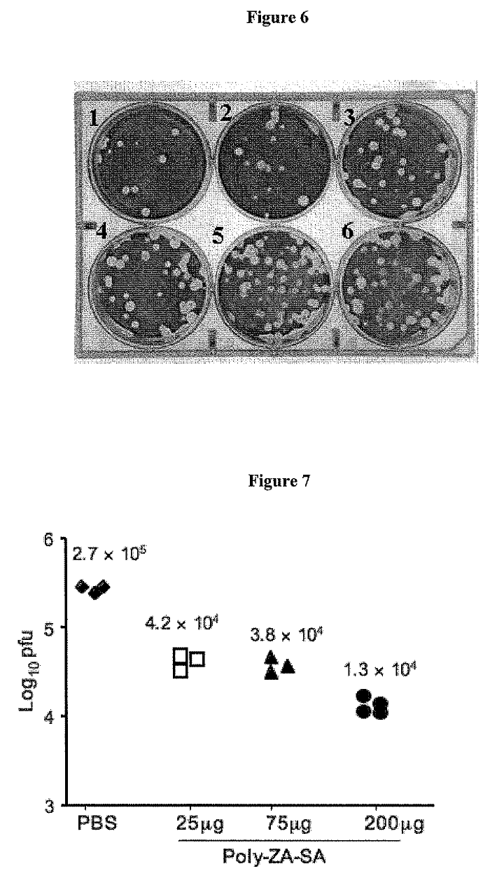

In a pilot study, a bifunctional polymer containing 10% sialic acid and 10% zanamivir was prepared and its IC₅₀ value was measured using the plaque reduction assay. The results are shown in FIG. 6 and Table 3.

TABLE 3
IC₅₀ values of various PIBMA-ZA-SA derivatives

| | IC₅₀ | | |
|---|---|---|---|
| | PIMBA | Sugar | |
| Compounds | (ng/ml) | (ng/ml) | (nM) |
| PIBMA | (2.1 ± 0.5) × 10⁴ | — | — |
| Sialic acid | — | >>5 × 10⁵ | >>10⁶ |
| derivative | | | |
| Zanamivir | — | (4.4 ± 0.5) × 10² | (6.3 ± 0.8) × 10² |
| derivative | | | |
| PIBMA- | 9.2 ± 5.4 | 3.3 ± 2 | 7 ± 4 |
| ZA(10%)- | | | |
| SA(10%) | | | |

As shown in Table 3, the IC₅₀ value for PIBMA-ZA-SA is 7 mM, which is a 90-fold increase compared to monomeric zanamivir derivative.

PIBMA-SA, PIBMA-ZA, PIBMA-SA-ZA and a combination of PIBMA-SA and PIBMA-ZA were also tested against human influenza A (A/Wuhan/359/95 (H3N2) and its mutant version that is resistant to oseltamivir) and influenza B (B/Hong-Kong/36/05, mutant strain, which is both resistant to zanamivir and oseltamivir). As shown in Table 4, PIBMA-SA is >10²-10³ fold more active than sialic acid derivative (monomer) against both influenza A and influenza B. The IC₅₀ values of PIBMA-ZA, are 77 nM and 250 nM against the wild type and mutant strains of influenza A viruses, respectively, which are much lower than those for the zanamivir derivative, 13 μM and 120 μM.

An equimolar combination of the monofunctional agents (PIBMA-SA+PIBMA-ZA) is at least an order of magnitude more potent inhibitor of influenza A viruses, whether of the wild-type or mutant strains, than the best monofunctional multivalent agent, namely PIBMA-ZA alone (and even more so compared to PIBMA-SA alone), indicating that the effect is more than additive. A similarly marked enhancement of the antiviral potency could be achieved with PIBMA-ZA(10%)-SA(10%) i.e., equimolar sialic acid derivative and zanamivir derivative covalently bonded to the same poly(isobutylene-alt-maleic anhydride) chain. It is noteworthy that the bifunctional polymer-attached ligands, both (PIBMA-SA+PIBMA-ZA) and PIBMA-ZA-SA, are still ˜10 times more potent than PIBMA-ZA (Table 4) against influenza A (Wuhan strains). Whereas in case of influenza B, PIBMA-ZA exhibited best antiviral activity (IC₅₀=41 nM).

TABLE 4
IC₅₀ values of various PIBMA derivatives against human influenza A
(wild type and mutant strains) and influenza B (mutant strain).

| | IC₅₀ (nM) | | |
|---|---|---|---|
| | Wuhan | Wuhan | Hong-Kong |
| Compounds | (Wild-type) | (Mutant) | (Mutant) |
| Sialic acid | (8.3 ± 1.5) × 10⁵ | >>10⁶ | >10⁶ |
| derivative | | | |
| PIBMA-SA | (2.9 ± 1.6) × 10² | (4.1 ± 0.7) × 10² | (3.8 ± 2.5) × 10³ |
| (10%) | | | |
| Zanamivir | ND | ND | (2.1 ± 1.8) × 10⁵ |
| Zanamivir | (1.3 ± 0.3) × 10⁴ | (1.2 ± 0.5) × 10⁵ | (2.4 ± 1.6) × 10⁵ |
| derivative | | | |
| PIBMA-ZA | 77 ± 25 | (2.5 ± 1.3) × 10² | 41 ± 22 |
| (8%) | | | |
| PIBMA-SA | 3.5 ± 1.6 | 23 ± 5.5 | (2.8 ± 2.5) × 10² |
| (10%) + | | | |
| PIBMA-ZA | | | |
| (8%) | | | |
| PIBMA-ZA | 8.4 ± 6.3 | 16 ± 11 | (6.6 ± 2.0) × 10² |
| (10%) − SA | | | |
| (10%) | | | |

PIBMA-SA, PIBMA-ZA and PIBMA-SA-ZA were also tested against avian influenza A virus (A/Turkey/MN/833/80 (H4N2) and its mutant version that is resistant to zanamivir). As shown in Table 5, the Sialic acid derivative did not show any antiviral activity (no appreciable reduction of the number of plaques compared to control even at a 10⁶ nM concentration) whereas IC₅₀ values of PIBMA-SA were 32 μM and 89 μM against the wild type and mutant strains, respectively. The IC₅₀ values of PIBMA-ZA were 3 μM and 31 μM against wild-type and mutant strains respectively, which are 4-fold and 17-fold lower than those for the zanamivir derivative, PIBMA-ZA exhibited the most effective antiviral activity among the PIBMA derivatives.

TABLE 5
IC₅₀ values of various PIBMA derivatives against avian influenza A virus

IC₅₀ (nM)

| | Turkey | Turkey |
|---|---|---|
| Compounds | (Wild-type) | (Mutant) |
| Sialic acid derivative | >>10⁶ | >>10⁶ |
| PIBMA-SA (10%) | (3.2 ± 2.3) × 10⁴ | (8.9 ± 2.4) × 10⁴ |
| Zanamivir derivative | (1.2 ± 0.7) × 10⁴ | (5.1 ± 1.4) × 10⁵ |
| PIBMA-ZA (8%) | (3.0 ± 1.1) × 10³ | (3.1 ± 0.2) × 10⁴ |
| PIBMA-ZA (10%)- | (2.0 ± 1.4) × 10⁴ | (9.8 ± 2.5) × 10⁴ |
| SA(10%) | | |

To determine the efficacy of PIBMA-ZA-SA in inhibiting influenza virus infection in vivo, a mouse model of influenza virus infection was developed. The results are shown in FIG. 7. Mice at 8-12 weeks of age were administered, intranasally, 50 μl of PBS containing 25, 75, or 200 μg of PIBMA-ZA-SA. As controls, mice were given just PBS. After four hours, the mice were infected with 12,000 pfu of influenza virus A/Victoria/3/75 intranasally. Twenty-four hours after infection, the mice were sacrificed and virus titters in the lung homogenates were measured using the plaque formation assay. In the PBS-treated mice, the virus titer in the lung was 2.7×10⁵ pfu/mouse. In contrast, the lung virus titer was reduced approximately 7 to 20 fold in the mice that were given PIBMA-ZA-SA once in a dose-dependent manner. These results suggest that PIBMA-ZA-SA is effective at inhibiting influenza virus infection in vivo.

We claim:

1. A virucidal composition comprising two or more antiviral agents coupled to one or more water-soluble, biocompatible polymers.

2. The composition of claim 1, wherein the two or more viral agents are selected from the group consisting of zanamivir, sialic acid, oseltamivir, amantadine, rimantadine, and combinations thereof.

3. The composition of claim 1, wherein the two or more antiviral agents are covalently coupled to the same biocompatible polymer.

4. The composition of claim 1, wherein the water-soluble biocompatible polymer is selected from the group consisting of poly(isobutylene-alt-maleic anhydride), poly(aspartic acid), poly(glutamic acid), polylysine, poly(acrylic acid), plyaginic acid, chitosan, carboxymethyl cellulose, carboxymethyl dextran, polyethyleneimine, and blends and copolymers thereof.

5. The composition of claim 4, wherein the water-soluble biocompatible polymer is poly(isobutylene-alt-maleic anhydride).

6. The composition of claim 1, wherein the molecular weight of the polymer is from 1,000 to 1,000,000 Daltons, preferably 10,000 to 1,000,000 Daltons.

7. The composition of claim 3, wherein the two or more antiviral agents are sialic acid and zanamivir and the water-soluble, biocompatible polymer is poly(isobutylene-alt-maleic anhydride).

8. The composition of claim 1, wherein the concentration of each of the two or more antiviral agents is from about 5% to about 25% by weight of the polymer.

9. The composition of claim 7, wherein the concentration of sialic acid and zanamivir is independently selected from the group consisting of 5% by weight of the polymer, 8% by weight of the polymer, 10% by weight of the polymer, 18% by weight of the polymer, and 25% by weight of the polymer.

10. The composition of claim 9, wherein the concentration of sialic acid is 10% by weight of the polymer and the concentration of zanamivir is 10% by weight of the polymer.

11. The composition of claim 1, wherein the composition comprises a physical mixture of a first water-soluble, biocompatible polymer coupled to a first antiviral agent and a second water-soluble, biocompatible polymer coupled to a second antiviral agent.

12. The composition of claim 11, wherein the first and second polymers have the same chemical composition or different chemical compositions.

13. The composition of claim 11, wherein the first and second polymers have the same chemical composition.

14. The composition of claim 13, wherein the first and second antiviral agents are sialic acid and zanamivir and the polymer is poly(isobutylene-alt-maleic anhydride).

15. The composition of claim 14, wherein the concentration of the functionalized polymers in the mixture is equimolar.

16. The composition of claim 15, wherein the concentration of sialic acid and zanamivir is 10% by weight of the polymers.

17. A method of making a virucidal composition comprising two or more antiviral agents coupled to one or more water-soluble, biocompatible polymers, the method comprising coupling two or more antiviral agents or derivatives thereof to a water-soluble biocompatible polymer.

18. The method of making a virucidal composition comprising a physical mixture of a first water-soluble, biocompatible polymer coupled to a first antiviral agent and a second water-soluble, biocompatible polymer coupled to a second antiviral agent, the method comprising coupling a first antiviral agent to a first water-soluble, biocompatible polymer, coupling a second antiviral agent to a second water-soluble, biocompatible polymer, and mixing or blending the polymers together.

19. A method of treating or preventing a viral infection, the method comprising administering to a patient in need thereof an effective amount a virucidal composition comprising two or more antiviral agents coupled to one or more water-soluble, biocompatible polymers, the method comprising coupling two or more antiviral agents or derivatives thereof to a water-soluble biocompatible polymer.

20. The method of claim 19, wherein the two or more antiviral agents are covalently coupled to the same biocompatible polymer.

21. The method of claim 19, wherein the composition comprises a physical mixture of a first water-soluble, biocompatible polymer coupled to a first antiviral agent and a second water-soluble, biocompatible polymer coupled to a second antiviral agent.

22. The method of claim 19, wherein the viral infection is selected from the group consisting of wild-type human or avian influenza, mutant human or avian influenza, respiratory syncythial virus, and combinations thereof.

23. A pharmaceutical composition comprising a virucidal composition comprising two or more antiviral agents coupled to one or more water-soluble, biocompatible polymers, the method comprising coupling two or more antiviral agents or derivatives thereof to a water-soluble biocompatible polymer.

24. The composition of claim 23, wherein the two or more antiviral agents are covalently coupled to the same biocompatible polymer.

25. The composition of claim 23, wherein the composition comprises a physical mixture of a first water-soluble, biocompatible polymer coupled to a first antiviral agent and a second water-soluble, biocompatible polymer coupled to a second antiviral agent.

26. The composition of claim 23, wherein the carrier is suitable for enteral administration.

27. The composition of claim 23, wherein the carrier is suitable for parenteral administration.
